(19) United States
(12) Patent Application Publication
(10) Pub. No.: US 2024/0237925 A1
(43) Pub. Date: Jul. 18, 2024

(54) Method of Data Analysis for Long-Term Blood Glucose Concentration Trend

(71) Applicant: National Taiwan University, Taipei (TW)

(72) Inventors: Chi-Kuang Sun, Taipei (TW); Xu-hao Ye, Taipei (TW); Tzung-Dau Wang, Taipei (TW)

(21) Appl. No.: 18/113,692

(22) Filed: Feb. 24, 2023

(30) Foreign Application Priority Data
Jan. 18, 2023 (TW) 112102256

(51) Int. Cl. A61B 5/145

(52) U.S. Cl. CPC A61B 5/14532

(57) ABSTRACT

A method of data analysis is provided. The method is used for finding a long-term trend of blood glucose concentration. The method builds a model for estimating long-term glycemic variability and long-term blood glucose trajectory. Based on single-erythrocyte-level glycated hemoglobin distribution, the glycemic variability is analyzed. A first analysis method is to give a number. The number shows the level of the historical glycemic variabilities. A second analysis method is to restore the blood glucose trajectory over the past 20 weeks. Based on the single-erythrocyte-level glycated hemoglobin distribution, the present invention easily assesses blood-glucose-related clinical information for about 150 days. Hence, an important complement is obtained for diabetes-related or glucose-monitoring-related clinical applications.

### TECHNICAL FIELD OF THE INVENTION

The present invention relates to analysis of blood glucose concentration; more particularly, to analyzing glycemic variability based on single-erythrocyte-level glycated hemoglobin distribution, where long-term glycemic variability and long-term blood glucose trajectory are estimated.

### DESCRIPTION OF THE RELATED ARTS

All existing glycated-hemoglobin-based glucose-estimation methods cannot provide any information regarding blood glucose fluctuations although can assess ˜3-month average blood glucose level. Evolving evidence indicates that variations in blood glucose levels are associated with diabetes-related complications independent of glycated hemoglobin fraction.

Devices such as continuous blood glucose monitoring allow people with diabetes to determine their blood glucose levels on a continuous basis and estimate blood glucose fluctuations. However, the sensors must be changed about every 2 weeks, and the cost related could limit their widespread use.

All these methods could not provide long-term blood-glucose-related information instantly. Hence, the prior arts do not fulfill all users' requests on actual use.

### SUMMARY OF THE INVENTION

The main purpose of the present invention is to analyze glycemic variability based on single-erythrocyte-level glycated hemoglobin distribution, where long-term glycemic variability and long-term blood glucose trajectory are estimated.

Another purpose of the present invention is to, based on the single-erythrocyte-level glycated hemoglobin distribution, easily assess blood-glucose-related clinical information for about 150 days, where an important complement is obtained for diabetes-related or glucose-monitoring-related clinical applications.

To achieve the above purpose, the present invention is a method of data analysis for long-term blood glucose concentration trend, comprising steps of: (a) configuring a data processing terminal to obtain a first glycated hemoglobin distribution (HbA1c distribution), where the first HbA1c distribution comes from different red blood cells of the same examinee; (b) based on the first HbA1c distribution, calculating an average glycated hemoglobin value (F₀); and, based on a glycated hemoglobin production model and the number of the red blood cells covered by the first HbA1c distribution, obtaining a time resolution (ΔT) to analyze a trend of glucose concentration; (c) based on an empirical formula with the average glycated hemoglobin value, calculating an estimated average glucose (eAg); based on the time resolution, generating a first blood glucose trajectory being stable; and using a red blood cell survival model and the glycated hemoglobin generation model with the eAg to derive a second HbA1c distribution; and (d) comparing the first HbA1c distribution with the second HbA1c distribution to obtain a glucose variability (GV); based on the time resolution, changing blood glucose concentrations at different time periods in the first blood glucose trajectory to obtain and fit a third HbA1c distribution; and using the red blood cell survival model and the glycated hemoglobin generation model with differences between the third HbA1c distribution and the first HbA1c distribution being cumulated to a sum not reaching a maximum fitting error (α) to ultimately obtain a second blood glucose trajectory being a long-term blood glucose concentration trend, where, based on the GV and the long-term blood glucose concentration trend, glycemic inform-ation are provided to assist in medical diagnosis related to blood glucose. Accordingly, a novel method of data analysis for long-term blood glucose concentration trend is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according the present invention;

FIG. 2A˜FIG. 2Q are the views showing the blood glucose trajectories corresponding to the glycated hemoglobin distributions, separately;

FIG. 3A˜FIG. 3N are the estimation views showing the long-term glycemic variabilities and the long-term blood glucose trajectories; and

FIG. 4 is the estimation view showing the long-term glycemic variability and the long-term blood glucose trajectory of the state-of-use.

### DESCRIPTION OF THE PREFERRED EMBODIMENT

The following description of the preferred embodiment is provided to understand the features and the structures of the present invention.

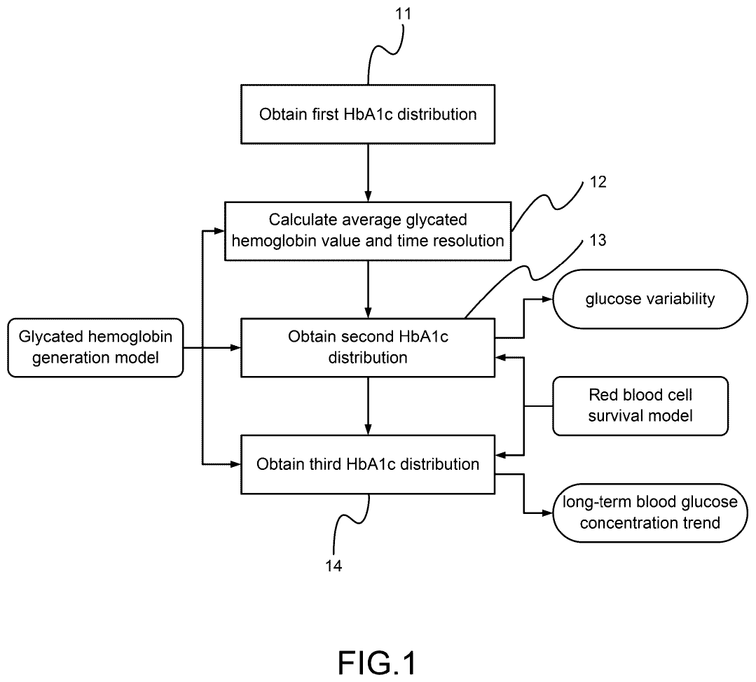

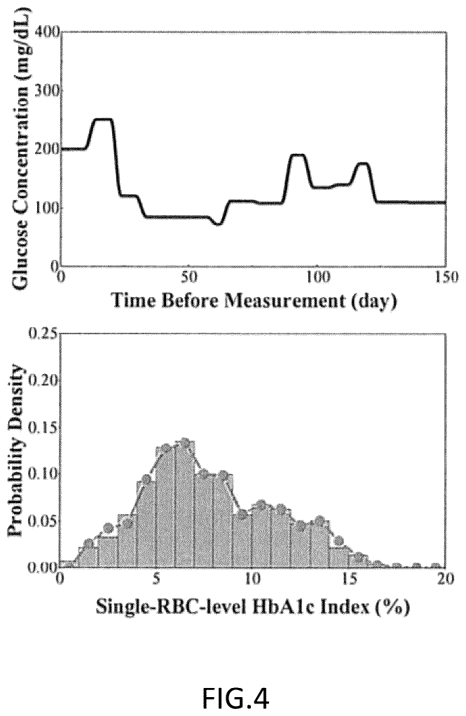

Please refer to FIG. 1˜FIG. 4, which are a flow view showing a preferred embodiment according the present invention; views showing blood glucose trajectories corresponding to glycated hemoglobin distributions, separately; estimation views showing long-term glycemic variabilities and long-term blood glucose trajectories; and an estimation view showing a long-term glycemic variability and a long-term blood glucose trajectory of a state-of-use. As shown in the figures, the present invention is a method of data analysis for long-term blood glucose concentration trend, comprising the following steps:
(a) Obtaining first HbA1c distribution 11: A data processing terminal is configured to obtain a first glycated hemoglobin distribution (HbA1c distribution), where the first HbA1c distribution comes from different red blood cells of the same examinee.
(b) Calculating average glycated hemo-globin and time resolution 12: Based on the first HbA1c distribution, an average glycated hemoglobin value (F₀) is calculated; and, based on a glycated hemoglobin production model and the number of the red blood cells covered by the first HbA1c distribution, a time resolution (ΔT) is obtained to analyze a trend of glucose concentration. Therein, the glycated hemoglobin generation model is expressed as a formula as follows:

$$HbA 1 c ( t ) = {\int }_{0}^{t} {e}^{- {k}_{g} \cdot G ( τ ) \cdot ( τ + {T}_{0} )} , \qquad ( 1 )$$

in which t is an age of red blood cells (cumulated time of blood circulation involved); HbA1c(t) is a glycated hemoglobin value of the red blood cells aged t; k_{g} is a constant and k_{g}=6.06×10⁻⁶ dL/mg/day; G(τ) is a blood glucose concentration of the red blood cells aged t on entering blood circulation; and T₀ is an equivalent residence time of the red blood cells in bone marrow.
(c) Obtaining second HbA1c distribution 13: Based on an empirical formula with the average glycated hemoglobin value, an estimated average glucose (eAg) is calculated; based on the time resolution, a first blood glucose trajectory being stable is generated; and a red blood cell survival model and the glycated hemoglobin generation model are used with the eAg to derive a second HbA1c distribution being stable. Therein, the empirical formula and the red blood cell survival model are respectively expressed as formulas as follows:

$$eAG = {28.7}^{*} {F}_{0} - 46.7 mg / dL , \qquad ( 2 )$$

and

$$β ( t ) = {e}^{- {( \frac{t}{b} )}^{a}} , \qquad ( 3 )$$

in which t is an age of red blood cells (cumulated time of blood circulation involved); β(t) is a survival rate of the red blood cells participating in blood circulation for a time of t; a is a constant of 5.58; and b is a constant of 125.63.
(d) Obtaining third HbA1c distribution 14: The first HbA1c distribution is compared with the second HbA1c distribution to obtain a glucose variability (GV); based on the time resolution, blood glucose concentrations at different time periods in the first blood glucose trajectory are modified to obtain and fit a third HbA1c distribution; and the red blood cell survival model and the glycated hemoglobin generation model are used with a cumulated sum of differences between the third HbA1c distribution and the first HbA1c distribution as the sum is not reaching a maximum fitting error (α), i.e. less than 0.1, to ultimately obtain a second blood glucose trajectory being a long-term blood glucose concentration trend. Based on the GV and the long-term blood glucose concentration trend, glycemic information are provided to assist in medical diagnosis related to blood glucose. Thus, a novel method of data analysis for long-term blood glucose concentration trend is obtained.

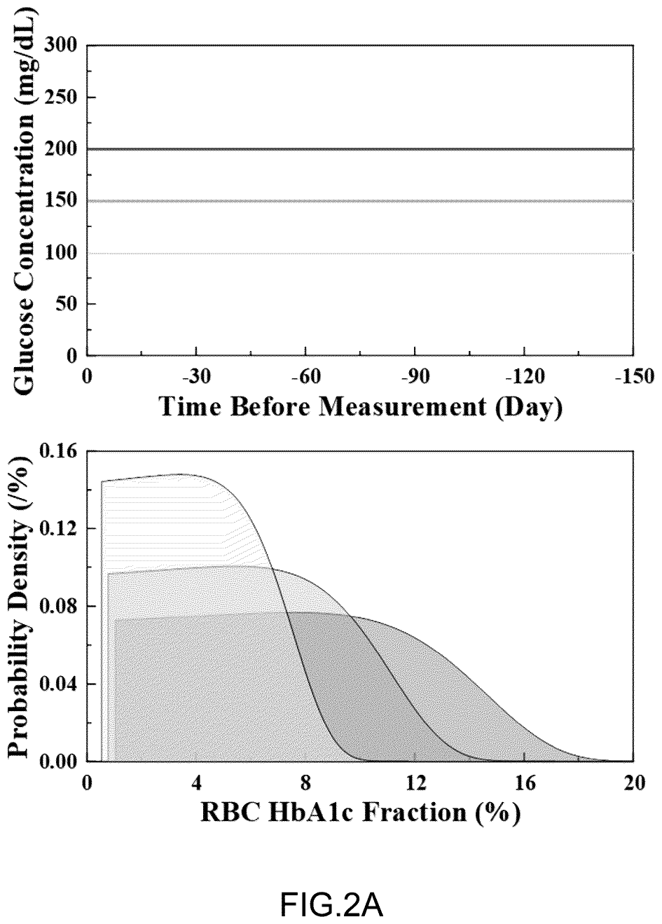

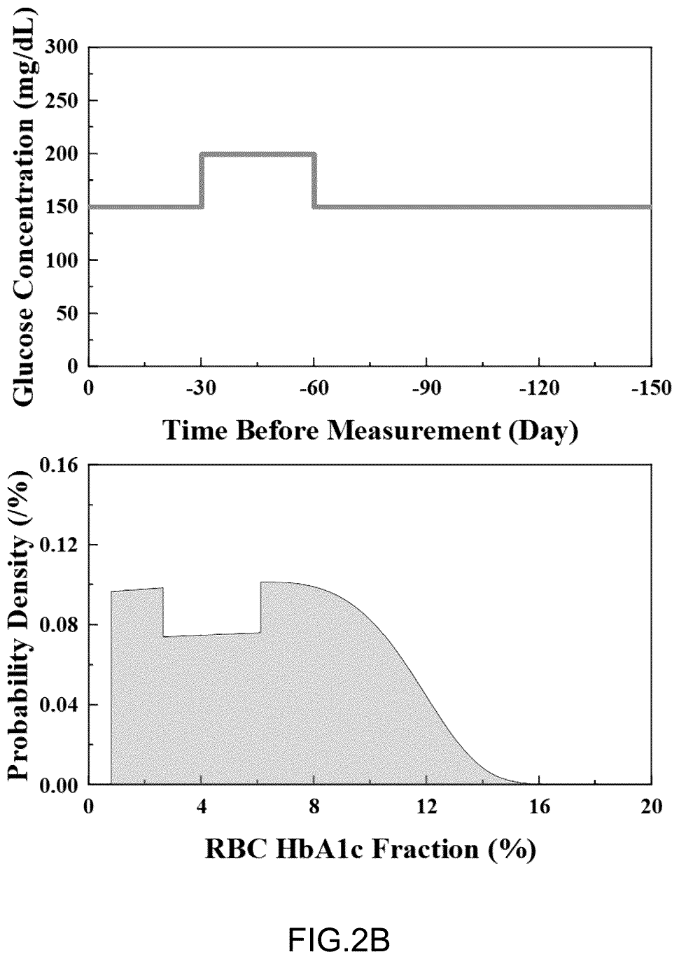

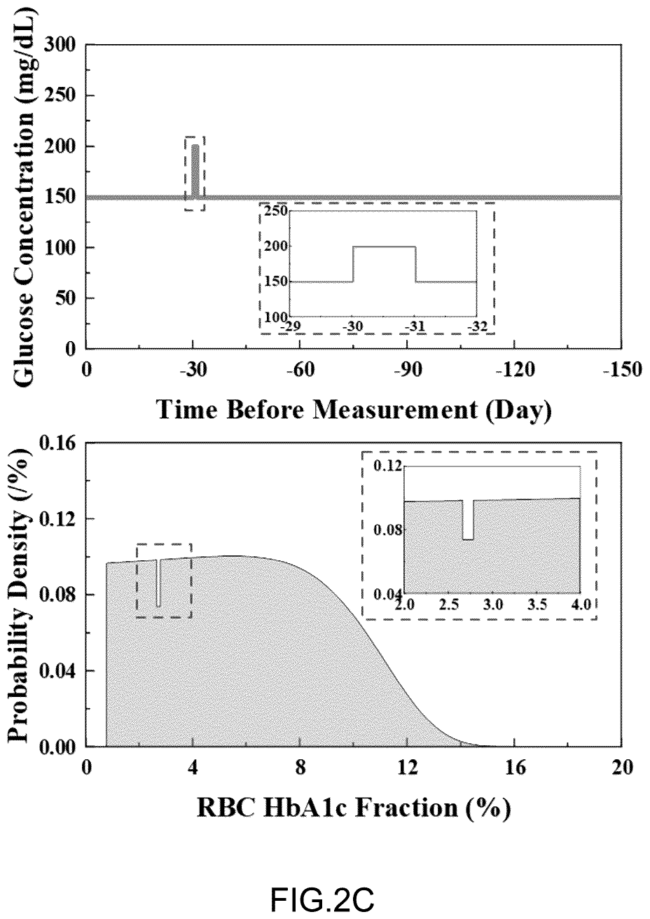

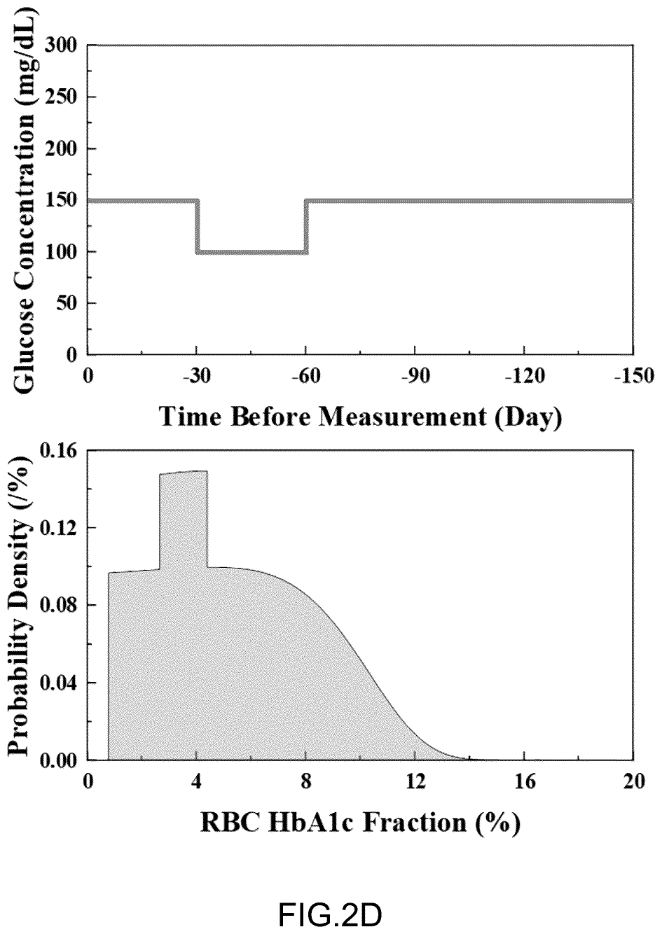

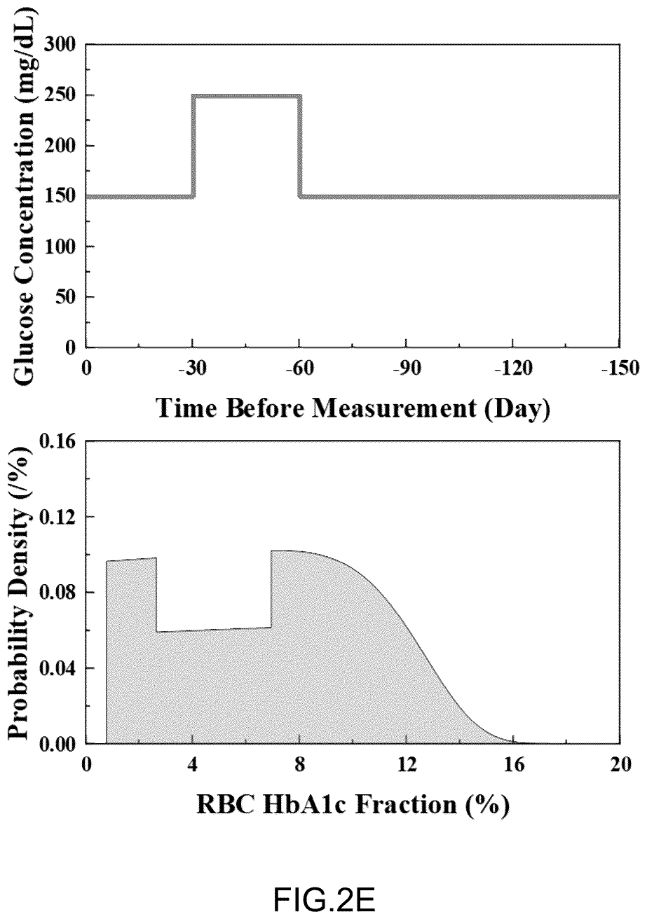

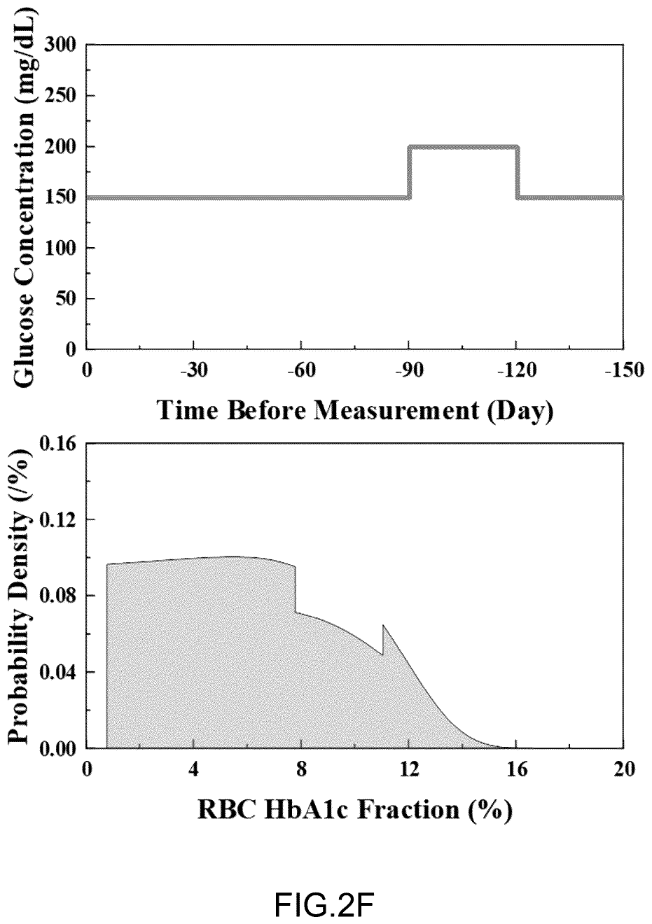

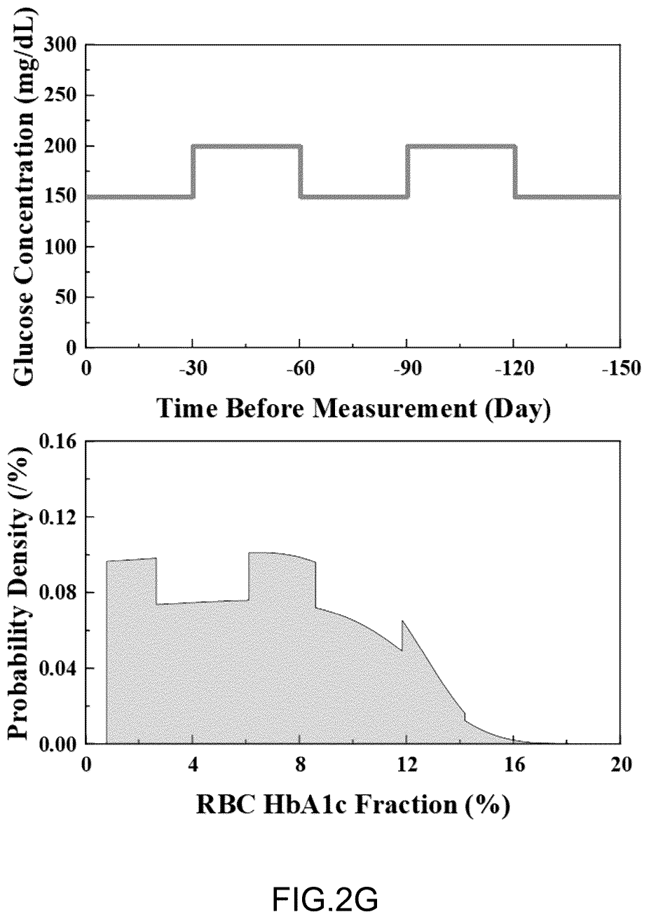

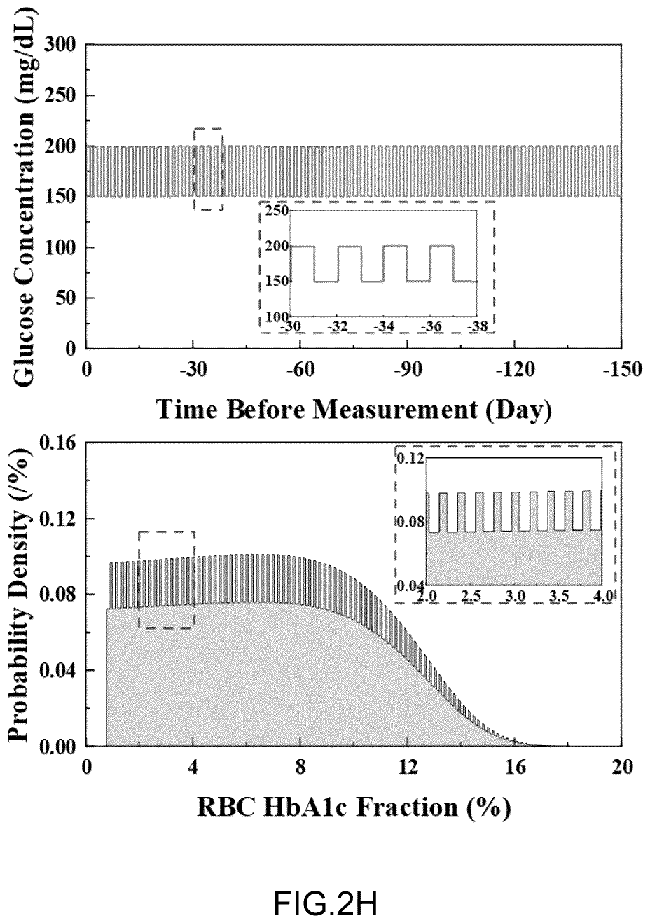

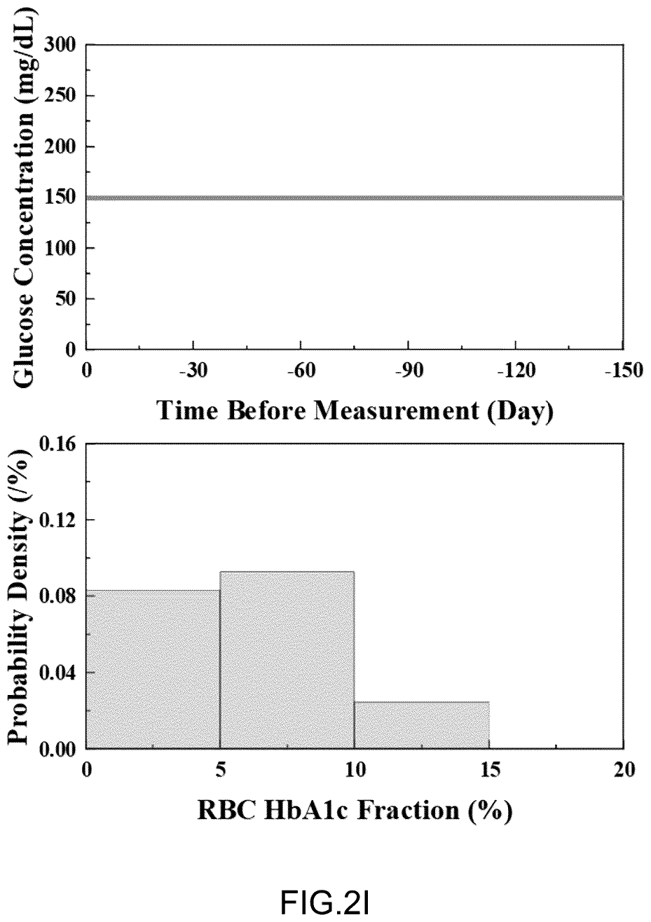

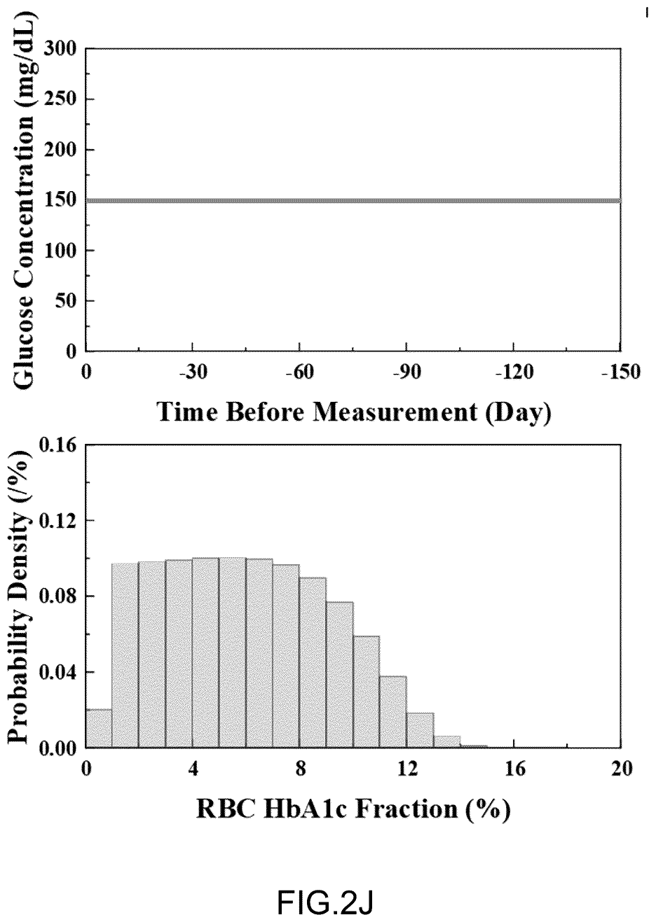

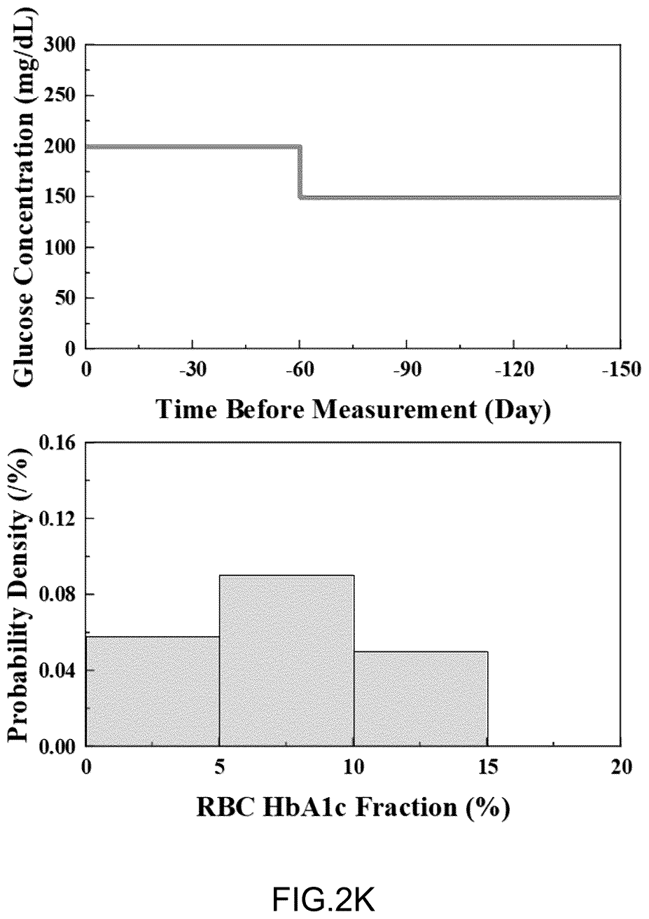

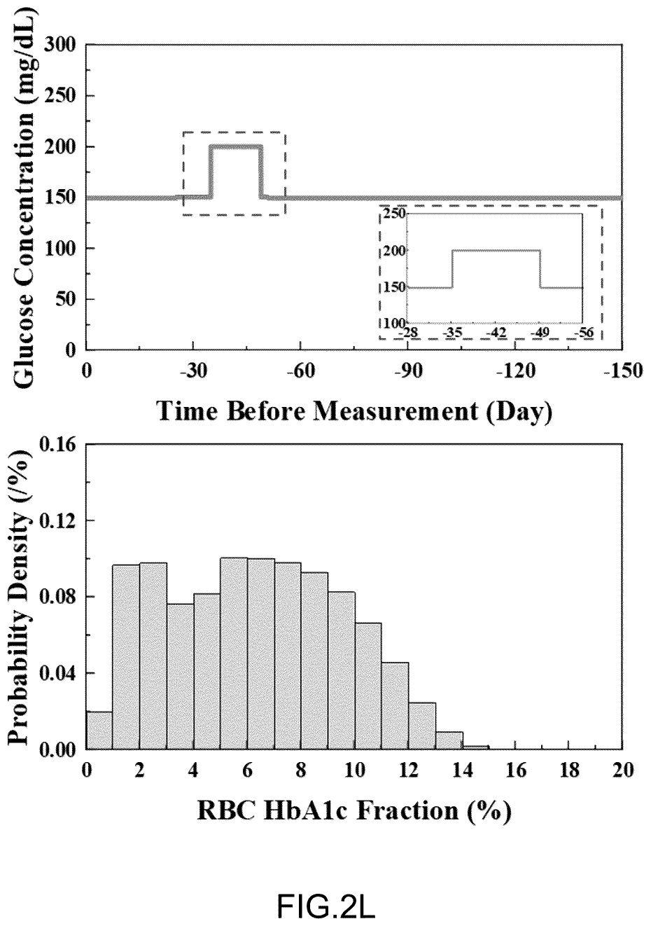

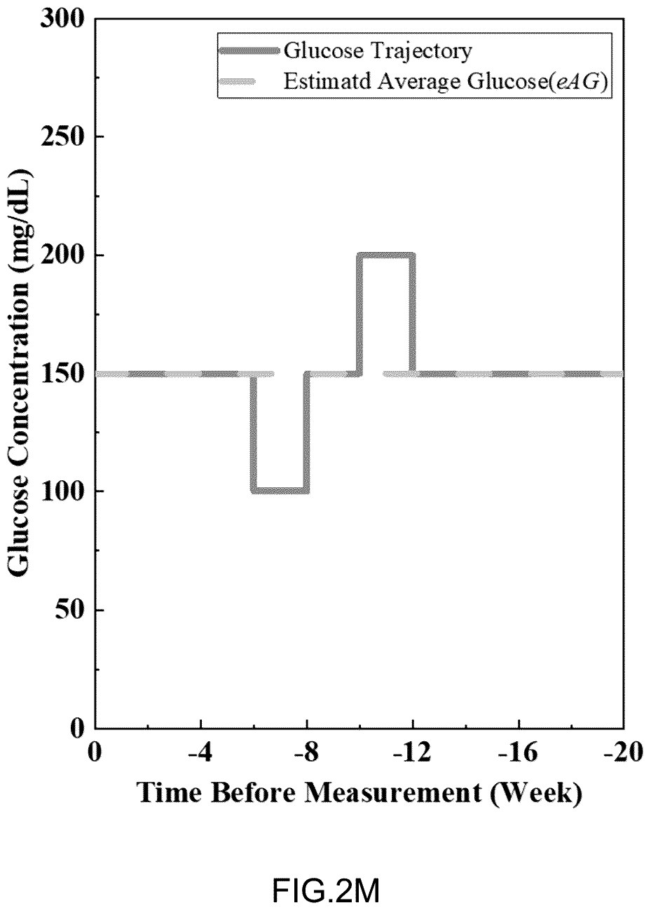

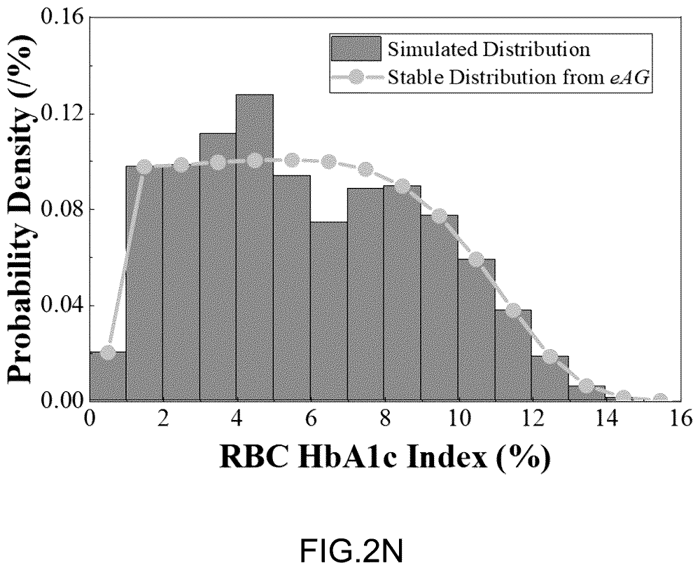

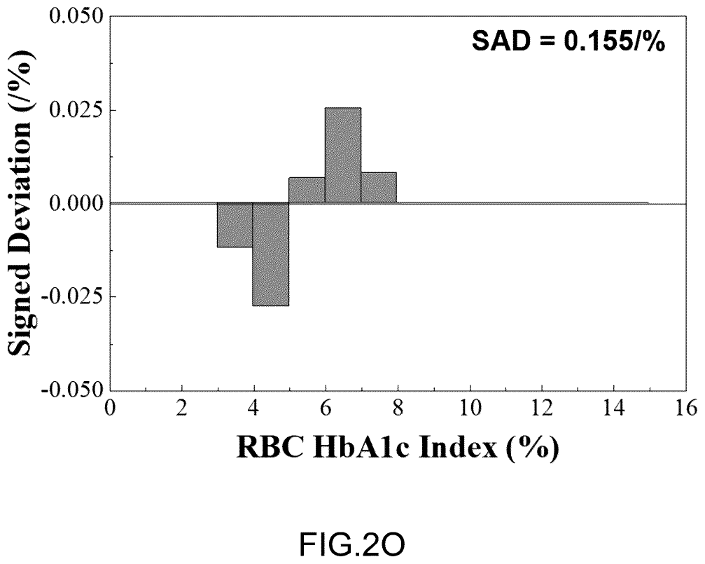

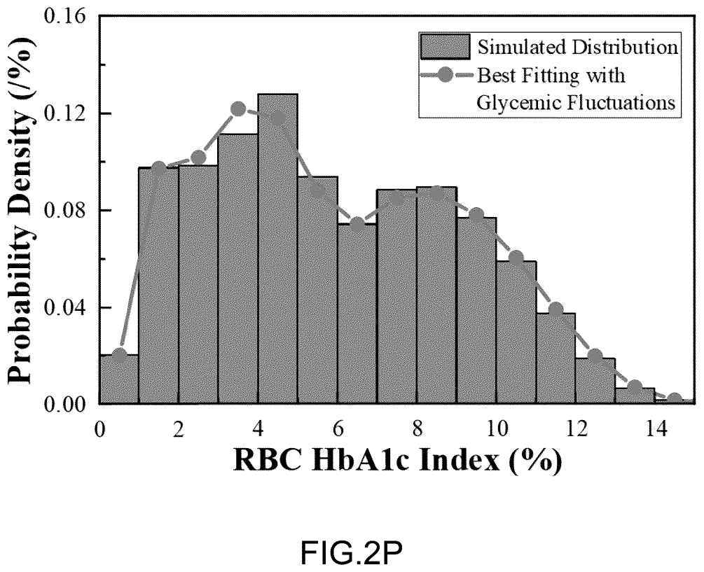

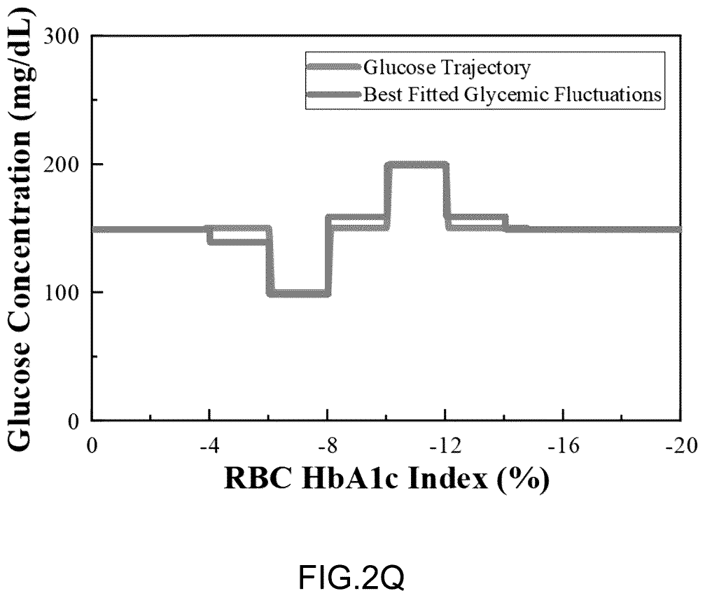

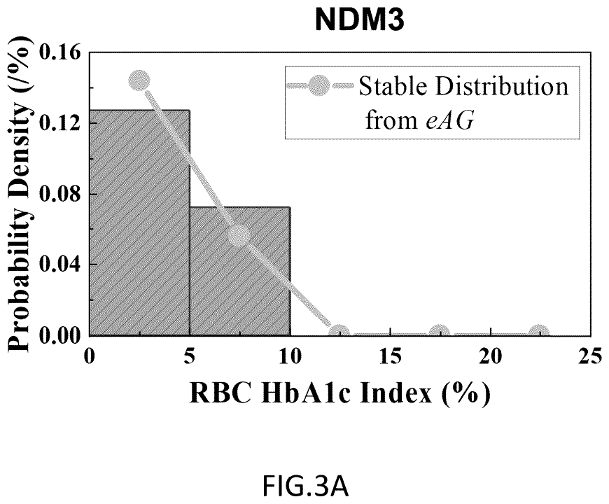

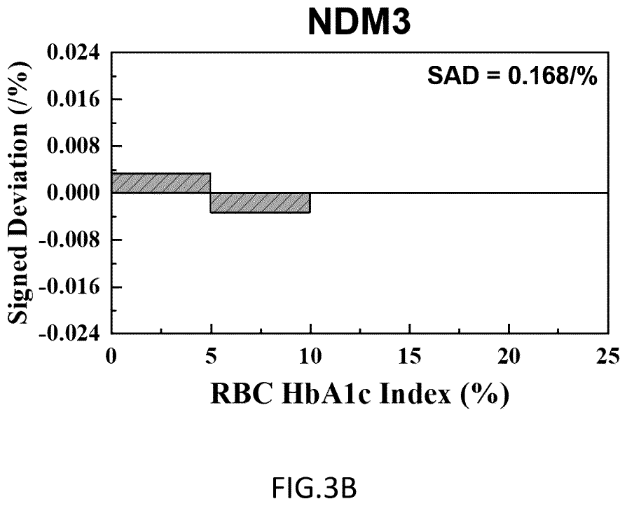

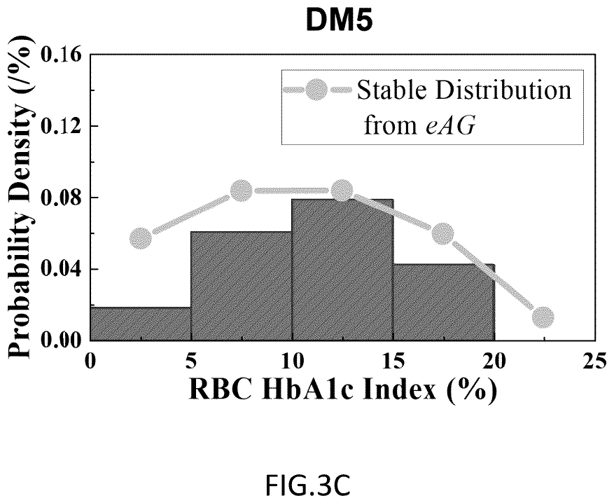

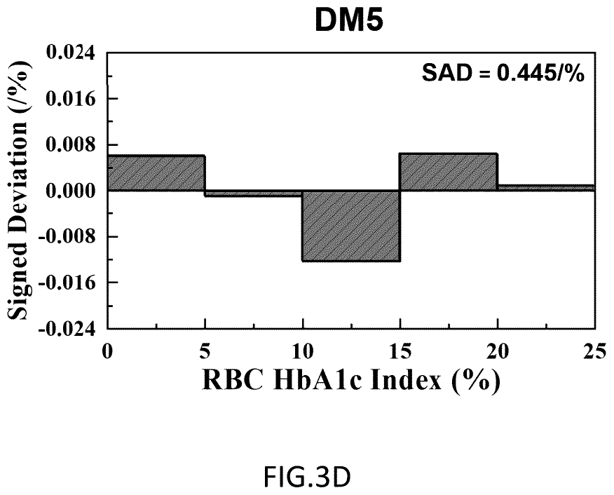

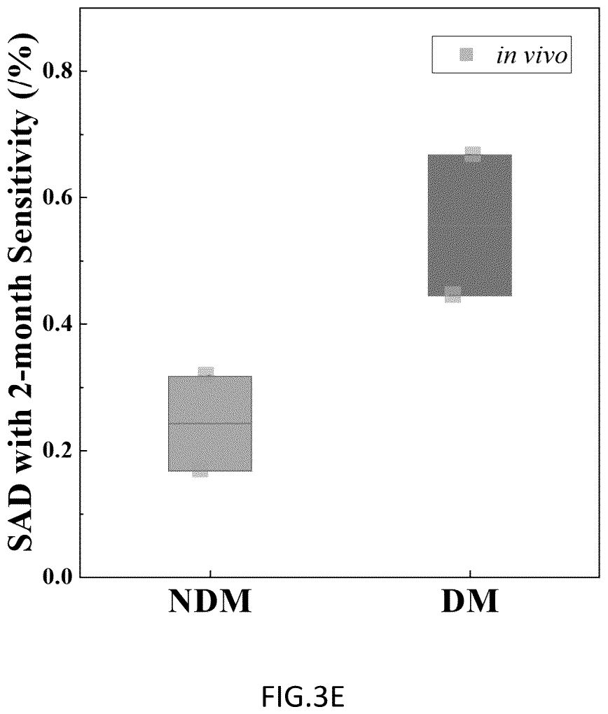

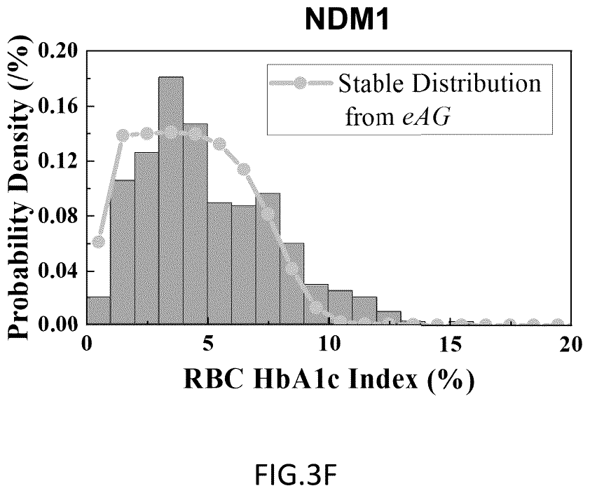

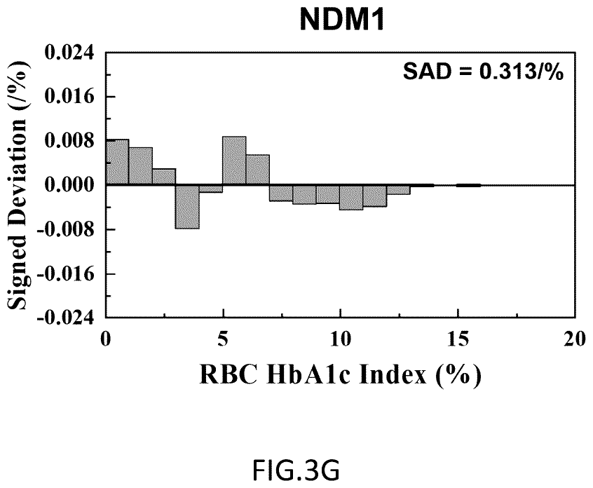

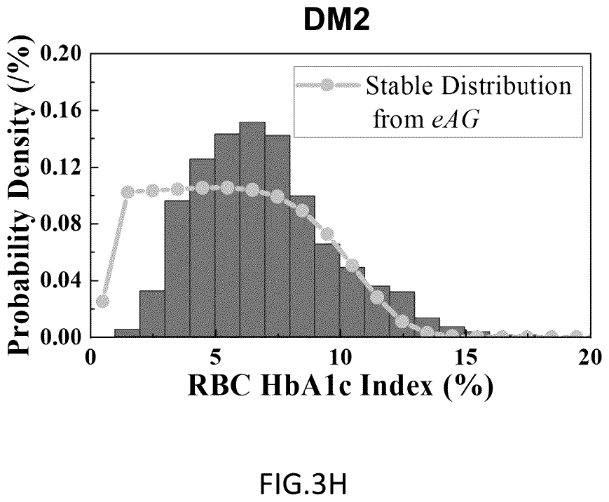

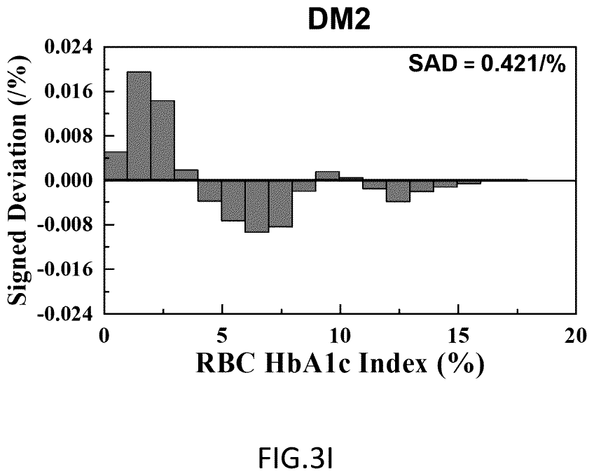

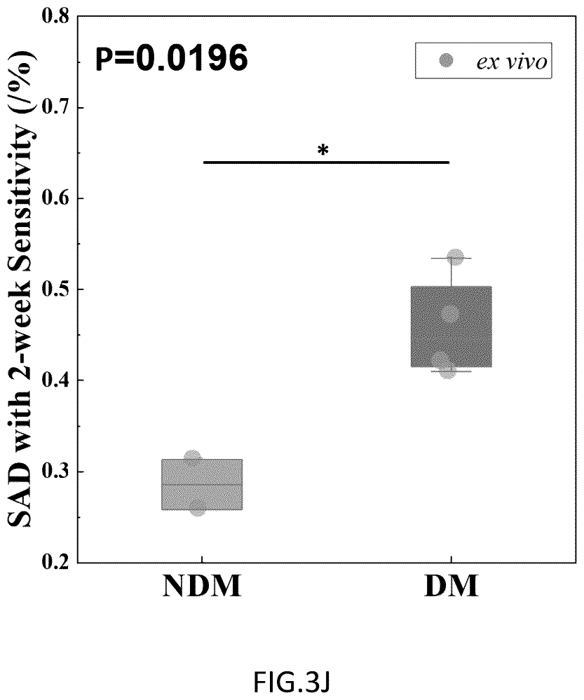

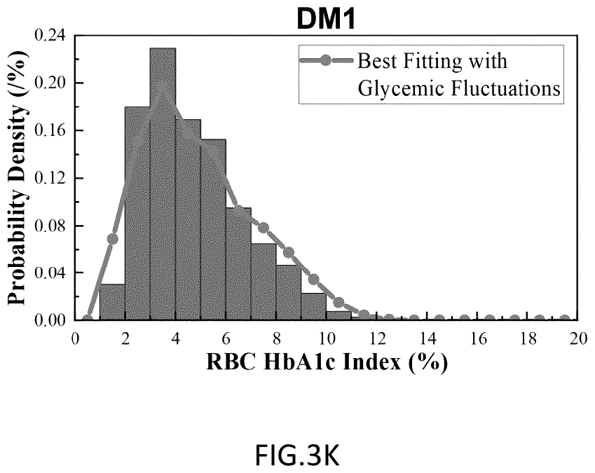

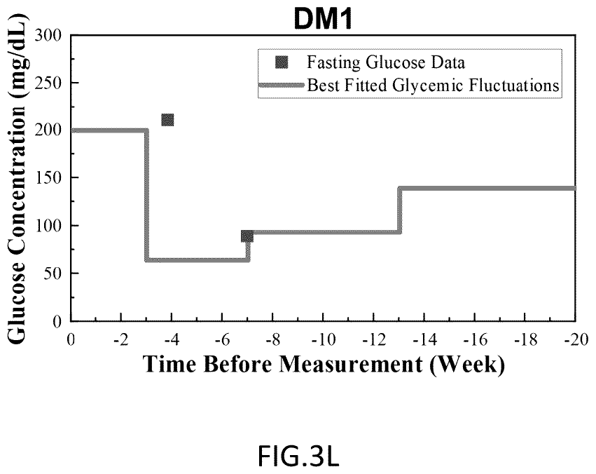

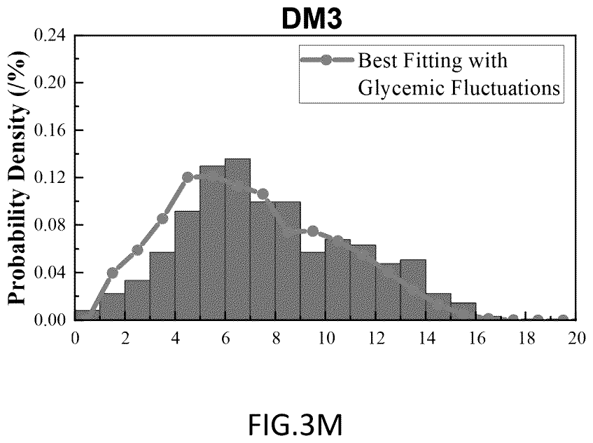

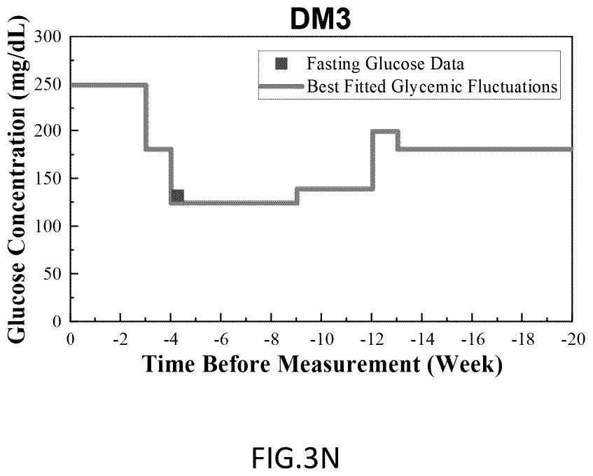

On using the present invention, the method according to the present invention builds a model for estimating long-term glycemic variability and long-term blood glucose trajectory (with blood glucose trajectories corresponding to glycated hemoglobin distributions shown in FIG. 2A˜ FIG. 2Q, separately), where, based on single-erythrocyte-level glycated hemoglobin distri-bution, glycemic variability is analyzed. There are two analysis methods, and the first method is to give a number. The number shows the level of the glycemic variability in history, as shown in FIG. 3A˜FIG. 3J. The second method is to restore the blood glucose trajectory over the past 20 weeks, as shown in FIG. 3K˜FIG. 3N (a similar result of continuous blood glucose monitoring with different time resolution).

In a state-of-use as shown in FIG. 4, the lower part shows the measured single-erythrocyte-level glycated hemoglobin distri-butions (light red bar) and the optimal fitting distributions obtained by using the model according to the present invention; and the upper part shows an estimate of blood glucose trajectory for 150 days based on the model. Hence, it is found that the model built by using the method according to the present invention is an important complement for glucose-monitoring-related clinical applications to solve the problem of estimating long-term glycemic variability and long-term blood glucose trajectory during monitoring diabetes.

To sum up, the present invention is a method of data analysis for long-term blood glucose concentration trend, where long-term glycemic variability and long-term blood glucose trajectory are estimated; based on the single-erythrocyte-level glycated hemoglobin distribution, blood-glucose-related clinical information for about 150 days are easily assessed; and, hence, an important complement is obtained for diabetes-related or glucose-monitoring-related clinical applications.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of data analysis for long-term blood glucose concentration trend, comprising steps of:
(a) configuring a data processing terminal to obtain a first glycated hemoglobin distribution (HbA1c distribution), wherein said first HbA1c distribution comes from different red blood cells of the same examinee;
(b) based on said first HbA1c distribution, calculating an average glycated hemoglobin value (F₀); and, based on a glycated hemoglobin production model and the number of said red blood cells covered by said first HbA1c distribution, obtaining a time resolution (ΔT) to analyze a trend of glucose concentration;
(c) based on an empirical formula with said average glycated hemoglobin value, calculating an estimated average glucose (eAg); based on said time resolution, generating a first blood glucose trajectory being stable; and using a red blood cell survival model and said glycated hemoglobin generation model with said eAg to derive a second HbA1c distribution; and
(d) comparing said first HbA1c distribution with said second HbA1c distribution to obtain a glucose variability (GV); based on said time resolution, modifying blood glucose concentrations at different time periods in said first blood glucose trajectory to obtain and fit a third HbA1c distribution; and using said red blood cell survival model and said glycated hemo-globin generation model with a cumulating sum of differences between said third HbA1c distribution and said first HbA1c distribution as said sum is not reaching a maximum fitting error (α) to ultimately obtain a second blood glucose trajectory being a long-term blood glucose concentration trend,
wherein, based on said GV and said long-term blood glucose concentration trend, glycemic information are provided to assist in medical diagnosis related to blood glucose.

2. The method according to claim 1,
wherein, in step (c), said eAg is obtained through an empirical formula as follows:
$$eAG = {28.7}^{*} {F}_{0} - 46.7 mg / dL .$$

3. The method according to claim 1,
wherein said red blood cell survival model is obtained as a formula as follows:
$$β ( t ) = {e}^{- {( \frac{t}{b} )}^{a}} ,$$
in which t is an age of red blood cells (cumulated time of blood circulation involved); β(t) is a survival rate of said red blood cells participating blood circulation for a time of t; a is a constant of 5.58; and b is a constant of 125.63.

4. The method according to claim 1,
wherein said glycated hemoglobin generation model is obtained as a formula as follows:
$$HbA 1 c ( t ) = {\int }_{0}^{t} {e}^{- {k}_{g} \cdot G ( τ ) \cdot ( τ + {T}_{0} )} ,$$
in which t is an age of red blood cells (cumulated time of blood circulation involved); HbA1c(t) is a glycated hemoglobin value of said red blood cells aged t; k_{g} is a constant and k_{g}=6.06×10⁻⁶ dL/mg/day; G(τ) is a blood glucose concentration of said red blood cells aged t on entering blood circulation; and T₀ is an equivalent residence time of said red blood cells in bone marrow.

5. The method according to claim 1,
wherein said maximum fitting error is 0.1.
